(19) United States
(12) Patent Application Publication
(10) Pub. No.: US 2024/0111028 A1
(43) Pub. Date: Apr. 4, 2024

(54) OPTICAL DEVICE AND METHOD FOR EXAMINING AN OBJECT

(71) Applicant: AKMIRA OPTRONICS GMBH, Potsdam (DE)

(72) Inventor: Alexander KNÜTTEL, Potsdam (DE)

(73) Assignee: AKMIRA OPTRONICS GMBH, Potsdam (DE)

(21) Appl. No.: 18/246,054

(22) PCT Filed: Sep. 17, 2021
(86) PCT No.: PCT/EP2021/075619

§ 371 Date: Nov. 7, 2023

(30) Foreign Application Priority Data
Sep. 21, 2020 (DE) 10 2020 124 521.1

(51) Int. Cl. G01S 7/481; A61B 1/00; G01S 17/66; G01S 17/894; G06T 7/50

(52) U.S. Cl. CPC G01S 7/4817; A61B 1/00009; A61B 1/00096; G01S 7/4816; G01S 17/66; G01S 17/894; G06T 7/50; G06T 2207/10028; G06T 2207/10068

(57) ABSTRACT

The present invention relates to an optical device for examining an object (16), comprising: a housing (24); an optical unit (26), arranged in the housing (24), for incident light; a sensor unit (28) having at least one image sensor (30, 32) arranged in the housing (24); a data processing unit (34) which is coupled to the sensor unit (28) and evaluates image signals from the at least one image sensor (30, 32); an illumination unit (42), arranged at least partially on or in the housing (24), for emitting grid light toward the object (16), wherein: by means of grid light reflected by the object (16) and reference light inside the housing, a three-dimensional point grid and associated reference datasets indicative of lateral information and depth information are provided using digital optical holography; by means of the device (10; 100; 120; 140), a relative movement of the device (10; 100; 120; 140) and the objective (16) is sensed, and associated movement information in the lateral and/or depth direction is created; object light emanating from the object (16) is sensed and, in temporal succession, a respective image dataset (35) is created which is registered relative to the reference dataset in order to create a 3D surface dataset; overlapping regions of two or more successive image datasets (35) are identified on the basis of the movement information, and the overlapping regions are smoothed by integrating the associated image signals, in particular without loss of detail. The invention also relates to a method.

The present invention relates to an optical device and method for inspecting an object with particularly visible object light to generate a 3D surface data set of the object, wherein the data set comprises lateral information and depth information. The device is preferably hand-held and/or hand-guided, wherein it is structurally compact. For example, the device is or forms and/or is comprised of an endoscopic device, a portable communication device or a head-mounted device.

The invention relates to the field of digital imaging and processing in which-processing in which examination light is incident on a single pixel image sensor of a sensor unit. Image signals from the image sensor are supplied to a data processing unit of the device and processed by the data processing unit to generate data sets. A display unit for displaying image information of the data sets and/or a memory unit for storing the data sets can be provided.

It is known to generate two-dimensional images by means of conventional (digital) photography. Enlargements can be achieved with the application of refractive or reflective optics. The disadvantage is the required space, combined with an increase in the dimensions of the device. The resolution is determined by the numerical aperture.

To provide a 3D surface data set with depth information, stereoscopy can be used. Here, two images of the scene are taken at different angles. A disadvantage is that a large distance between the image sensors (baseline) is required to achieve a high depth resolution. Regardless of this, artifacts can occur, for example in the case of edge gradients.

Depth information can also be provided, for example, using plenoptic camera systems. Multi-lens optics are used to generate wavefronts with different local phases, but this requires considerable computational effort to reconstruct the scene. Errors can occur particularly at low light incidence.

Devices and methods for generating digital image data sets are described, for example, in U.S. Pat. No. 10,536,684 B2, U.S. Pat. No. 8,456,517 B2, U.S. Pat. No. 6,664,529 B2 and U.S. Pat. No. 7,295,324 B2.

The objective of the present invention is to provide an optical device and method for examining an object with improved imaging characteristics.

This objective is solved by an optical device according to the invention for examining an object, comprising a casing, an optical unit for incident light arranged in the casing, a sensor unit with at least one image sensor arranged in the casing, a data processing unit which is coupled to the sensor unit and evaluates image signals of the at least one image sensor, an illumination unit arranged at least partially on or in the casing for emission of raster light in the direction of the object, wherein using raster light reflected from the object and reference light internal to the casing, a three-dimensional dot raster and reference data sets indicative thereof for lateral information and depth information are provided by digital optical holography, wherein a relative movement of the device and the object is detected via the device and movement information relating thereto in the lateral direction and/or depth direction is generated, wherein object light emanating from the object is detected and a respective image data set is generated successively in time which is registered relative to the reference data set for the creation of a 3D surface data set, wherein overlapping regions of two or more successive image data sets are identified on the basis of the movement information and the overlapping regions are smoothed by integration of the respective image signals, in particular without loss of detail.

In the device according to the invention, digital optical holography is used to generate lateral and/or depth information describing in particular the surface of the object. For this purpose, a dot raster can be emitted onto the object via raster light and fed to the at least one image sensor via the optical unit. Using reference light that interferes with the raster light on the image sensor, an optical grating can be “attached” to the object in this way, so to speak, which forms support points for the registration of the image data set. The image data set can be created by means of sub-scanning light, which is imaged onto the at least one image sensor via the optical unit. In particular, the image data set is preferably of higher resolution than the reference data set and is delimited only by the optical resolution of the system. The image data set can be computationally drawn over the optical grid of the reference data set, thereby creating a 3D surface data set. The device is in particular an optical 3D scanner or forms such a scanner.

In the device according to the invention, there is in particular the advantage that a relative movement of the device and the object can be detected by means of the device itself. Related movement information in lateral and/or depth direction can be provided. Based on the movement information, successive image data sets can be spatially matched. This is based on the consideration that the scene that can be observed by the device is also moved during a relative movement. It is therefore possible to identify overlapping regions of two or more image data sets. Noise inevitably contained in the image data sets can be reduced by integrating the image data sets and thereby smoothing the overlapping regions of the image data sets. This is preferably possible without losing object resolution and thus detail. As a result, the signal-to-noise ratio is improved (S/N ratio). In this way, the imaging quality of the device can be computationally improved with relatively little effort without loss of object resolution and thus spatial information, preferably up to the resolution limit, and a high-resolution 3D surface data set can be created. For example, high edge fidelity can preferably be achieved without loss of higher spatial frequencies.

Advantageously, the 3D surface data set, if noise-free or essentially noise-free, can be further processed using deconvolution algorithms to generate “super-resolution” images. Alternatively or complementarily, for example, “super-resolution” images are possible by interpolation within the 3D surface data set.

The invention includes the consideration that in particularly hand-held and/or hand-guided devices a relative movement to the object often already occurs due to hardly avoidable tremors of the user. Therefore, it is particularly possible to use the trembling movement itself as a basis for the evaluation of the movement information. In this context, it is also advantageous in particular that the movement information can be obtained by means of the device itself, the components of which are partly arranged in a common casing. This in turn favors a compact design of the device with the aim of hand holding and/or hand guiding.

In the case of the relative movement, it may be provided that this is limited to a purely lateral movement or to a movement solely in the depth direction (axial). However, the invention is not limited to this. In particular, a 3D relative movement may be recognized and applied.

“Smoothing” in the present case can be understood as integrating signal contributions from the overlapping regions of the two or more successive image data sets in time. Averaging may be provided wherein no “blurring” with loss of edge fidelity is performed in order to preserve object resolution without loss.

It is understood that the processing of image signals of the at least one image sensor, the creation and/or calculation of image data sets as well as the calculation of movement information can be performed by the data processing unit. This is not mentioned in detail below for ease of reading. However, it is emphasized that the data processing unit is designed and programmed to be able to perform the operations in this regard.

In particular, overlapping regions of the two or more image data sets may be mathematically superimposed by inverse movement. If the relative movement is known, subsequent image data sets can be computationally shifted back by an inverse amount and thus overlapped with a preceding image data set. The smaller the relative movement, the greater the overlap between two regions.

A single exposure time for a respective image data set is preferably smaller than an examination period over which the image data sets are integrated.

In practice, for example, a frame rate of about 250 Hz or more is advantageous, preferably up to about 1 kHz and above.

It is advantageous if a plurality of 3D surface data sets are combined to form an overall scene of the object, wherein boundaries between 3D surface data sets are determined based on the movement information. Over a longer time period, different lateral scenes can be stitched together. In contrast, such an overall scene cannot be created for individual images, particularly with limited aperture, in conventional devices. Preferably, the stitched individual scenes comprise no or only a small offset to each other.

It is advantageous if, in order to determine the relative movement, at least one luminous spot of the dot pattern with speckle pattern generated by the raster light on the object is examined as a function of time, wherein a displacement of the speckle pattern within a luminous spot is determined and movement information in the lateral direction is derived therefrom. On the surface of the object, which is typically not perfectly smooth in practice, a speckle pattern can be created via multiple interferences. If the luminous spot of the raster light moves relative to the object, the movement information in the lateral direction can be determined with high accuracy by correlating successive representations of the luminous spot with speckle pattern (speckle correlation), because the speckle pattern remains stationary relative to the object.

In particular, a plurality of luminous spots can preferably be examined synchronously. For example, the different spots can provide different lateral velocities for moving (“breathing”) objects. This makes the application of the device particularly interesting in the medical environment, intracorporeally and/or extracorporeally.

Conveniently, a deconvolution operation can be performed on the 3D surface dataset. This has already been discussed in the preceding.

For example, the raster light is or comprises infrared light and/or light of the visible spectrum. For example, a wavelength window of approximately 700 nm to 1300 nm is used for raster light. The spectrum of the raster light may extend into the visible spectrum to wavelengths less than 700 nm.

The raster light and the reference light are particularly coherent. For this purpose, the illumination unit preferably comprises a laser light source, which is preferably integrated in the casing. A beam splitter element can separate raster light and reference light for coupling into a respectively provided optical path, for example via waveguides.

It is understood that the reference light should comprise the same spectrum as the raster light.

The raster light and the reference light preferably comprise a spectrum of a plurality of discrete wavelengths. At least two wavelengths are provided here, preferably multiple wavelengths. For example, within a wavelength window of approximately 10 nm, multiple wavelengths are used approximately at nm intervals.

A movement information in depth direction and/or an absolute depth information is preferably determinable on the basis of phase differences of the multiple wavelengths. Due to the multiple wavelengths, there is preferably the advantage that an absolute depth information about the distance of the object from the device can be determined at the respective luminous spot of the dot grid. It is also advantageous if movement information in the depth direction can be determined on the basis of the phase differences of the multiple wavelengths of the raster light and the reference light.

The object light is or preferably comprises light of the visible spectrum, in particular of a continuous spectral range. For example, the object light is ambient light. Alternatively, quasi-monochromatic light can be used, for example with red, green and blue portions.

Preferably, the raster light and the object light comprise different spectra to enable separation of the object light and the raster light depending on wavelength and thereby simplify the evaluation.

The object light is, for example, non-coherent, in particular to avoid interference patterns (speckles) that are undesirable for observing the scene, or partially coherent, for example in the case of quasi-monochromatic light.

Advantageously, the illumination unit comprises at least one light guide for raster light in the casing and an optical uncoupling element arranged particularly distally on the casing. Thus, for example, a compact design can be achieved at least distally of the device. In contrast, the light source can comprise a large spatial distance from the uncoupling element. Such a design is suitable, for example, for endoscopic devices.

The light guide can comprise, for example, at least one rod or one optical fiber and accordingly be rigid and/or flexible. Conceivable is for example the application of a GRIN light guide (gradient index) to achieve spatial collimation.

The at least one light guide is guided, for example, along a casing wall of the casing, in particular on the inside of the casing wall. The casing wall is, for example, an outer wall.

The uncoupling element is or comprises, for example, a planar hologram, for fanning out the raster light as a dot raster onto the object.

The illumination unit advantageously comprises a light source for providing the raster light and the reference light, which is preferably arranged in the casing.

In a preferred embodiment of the invention, the illumination unit can be used to emit raster light with a time offset via different uncoupling elements and to provide the corresponding reference light. For example, raster light can be emitted respectively clocked via different uncoupling elements in order to generate different point rasters and to cover a larger area of the object. For example, raster light can be guided through different light guides to the distal end of the device, where it can be coupled out.

The illumination unit preferably comprises a light source for providing the object light, which is preferably arranged in the casing. In this way, the object can be selectively illuminated, thereby improving the signal-to-noise ratio.

Advantageously, the illumination unit comprises at least one light guide for object light in the casing and an optical uncoupling element arranged particularly distally on the casing. As mentioned in the preceding, for example, at least one rod and/or one fiber is used here.

In a preferred embodiment of the invention, a common image sensor is provided which is sensitive to the spectrum of the object light and to the spectrum of the raster light. This gives the possibility of a structurally simple design of the device with a compact design at the same time. In particular, only exactly one image sensor can be provided.

In another advantageous embodiment of the invention, the device comprises two image sensors, wherein object light can be guided to one of the image sensors and raster light can be guided to the other image sensor via an optical element of the optical unit.

The image sensors are preferably more sensitive to the respectively detected light than to the respectively other light. In particular, the quantum efficiency of a respective image sensor is as high as possible and preferably maximum for the respective light used, object light and/or raster light.

The preceding optical element is or preferably comprises at least one wavelength-sensitive beam splitter element, in particular a beam splitter cube or a beam splitter plate.

The optical unit conveniently comprises at least one polarizing element, particularly for the raster light and/or the reference light. For example, the beam splitter cube or beam splitter plate comprises a polarizing layer to reduce unwanted signal contributions and improve the signal-to-noise ratio.

Preferably, the optical unit comprises at least one filter element for reducing stray light, preferably arranged on the entrance side.

Advantageously, the optical unit may comprise a refractive optical element for the object light on the entrance side. The light-refracting op-tical element is for example a lens, for example a converging lens or a microlens array. The microlens array may be convexly curved, for example.

Two image sensors may be provided, which in a preferred embodiment of the invention are positioned side by side, in particular in a common plane, and covered by at least one entrance window. For example, a common entrance window is applied above the image sensors. By positioning the image sensors laterally next to each other, a flat design can preferably be achieved.

Accordingly, the device is preferably of flat design, and the entrance window is arranged on a surface of the casing along which the extension of the device is substantially greater than in a direction transverse thereto.

In a preferred embodiment of the invention, two image sensors can be provided which are arranged in planes aligned at an angle to one another, and a wavelength-sensitive beam splitter element is arranged upstream of a respective image sensor in the Direction of arrival of the object light or the raster light. This preferably allows a compact design of the device in three dimensions. The beam splitter element is, for example, a beam splitter cube or a beam splitter plate and is transmissive for one light (for example the raster light) and reflective for the other light (for example the object light). Preferably, the image sensors are immediately adjacent or arranged close to the beam splitter element. Preferably, the angle may be 90° or essentially 90°.

The optical unit advantageously comprises an image sensor sensitive to object light upstream in the direction of incidence of the object light a phase and/or amplitude modulating optical element. In this way, for example, superimposed images can be generated on the image sensor. For example, a microlens array can be used for multifocal imaging. The optical element can alternatively be, for example, a phase mask or an amplitude mask (U.S. Pat. No. 8,243,353 B1).

The at least one image sensor, in particular the image sensors, and/or the optical unit are advantageously arranged at a distal end or end segment of the device in order to achieve a compact design.

It is advantageous if the reference light is incident on the at least one image sensor with a planar or essentially planar wavefront.

The wavefront is preferably inclined relative to a plane of the at least one image sensor, with regard to an improved evaluation. It is particularly intended that the inclination depends on a wavelength of the reflection light. Accordingly, the wavefronts of different wavelengths preferably comprise different inclinations relative to the plane.

The optical unit preferably comprises an optical element for coupling out reference light inside the casing. For example, light from the light source is divided into raster light and reference light, which is coupled out via at least one light guide in the casing. Emission of reference light externally and subsequent coupling into the device can be omitted. This favors the compact design of the device.

The optical unit preferably comprises a VPH (volume phase hologram) for diffraction of the reference light in the direction of the at least one image sensor. For example, the planar wavefronts of different wavelengths of the reference light mentioned preceding can be diffracted via the VPH.

The VPH may in particular be or comprise a transmission or reflection grating. If the Bragg condition is satisfied, reference light can be diffracted at the grating in the direction of the image sensor.

In a preferred embodiment of the invention, the VPH is positioned at an entrance side of the device and is transmissive to the object light and/or the raster light. For example, the reference light is diffracted back by the VPH towards the image sensor where it interferes with the raster light.

Alternatively or additionally, the VPH can be aligned parallel to a plane of an image sensor and arranged immediately upstream of the image sensor in the direction of incidence of the raster light.

In a preferred embodiment of the invention, the illumination unit comprises a plurality of microlenses arranged side by side in a row, which generate an essentially planar wavefront of the reference light above a plane of the at least one image sensor. This lends itself, for example, to a low profile design of the device. Alternatively, a multi-volume hologram or an array of GRIN lenses may be provided, for example.

The optical unit conveniently comprises an optical element for expanding reference light with a planar or essentially planar world front in the direction of the at least one image sensor, wherein the optical element is, for example, a concave mirror. The optical element can be used in particular instead of the VPH.

Advantageously, the device according to the invention comprises a compact structure.

Assuming at least one image sensor with a square cross-sectional area of the surface a², wherein a is an edge length, the device advantageously comprises a cross-sectional area of less than 1.5 a², preferably less than 1.25 a², on the inlet side. Any components of the device (for example the optical unit and the casing) which project beyond the area of the image sensor can be designed to be specifically compact.

If a beam splitter element such as a beam splitter cube is used, its dimensions can correspond approximately to those of the image sensors. The optical components of the optical unit can thus preferably be accommodated in a volume of approximately a³.

Assuming at least one image sensor with a square cross-sectional area of the surface a², wherein a is an edge length, the device preferably comprises a height of approximately a/4 or less and a volume of a³/2 or less on the inlet side when one image sensor or two image sensors are present in flat design.

A sampling rate for recording the image data sets is preferably about 250 kHz or more, preferably up to about 1 kHz or more.

A respective spot size of the raster light on the object is preferably about 50 μm to 500 μm, preferably about 100 μm to 250 μm.

A working distance of the device from the object can vary, for example, from a few mm to a few hundred mm. For example, the working distance is about 50 mm to 100 mm.

The raster points of the raster light may preferably comprise a distance of about 1 mm from each other on the object, wherein the distance may depend on the working distance of the device from the object.

As mentioned above, the device is preferably hand-held and/or hand-guided.

In particular, the device may be an endoscopic device which is insertable, at least in sections, with the casing into the object to be examined. The examination object may be a human or animal body or a thing. An application in the dental field may be envisaged, wherein the device is wholly or partially insertable into the oral cavity.

The device may be used, for example, in industrial metrology.

The device is preferably a portable communication device or is comprised by such a device, in particular a smartphone or a tablet computer.

In an exemplary embodiment, the device is a head-mounted device, for example data glasses, specifically for AR (augmented reality) applications.

Accordingly, the present invention also relates to an endoscopic device, a smartphone or a tablet computer and/or a head-mounted device, comprising at least one device of the preceding type.

The above-mentioned objective is solved by a method according to the invention having the features of independent claim 25.

The advantages already mentioned in connection with the explanation of the device according to the invention can also be achieved by application of the method. The device features can be implemented according to the method.

Advantageous embodiments of the method result from advantageous embodiments of the device. In this respect, reference can be made to the preceding explanations.

The following description of preferred embodiments of the invention, which are in particular 3D scanners, serves in connection with the drawing to explain the invention in more detail. It shows in:

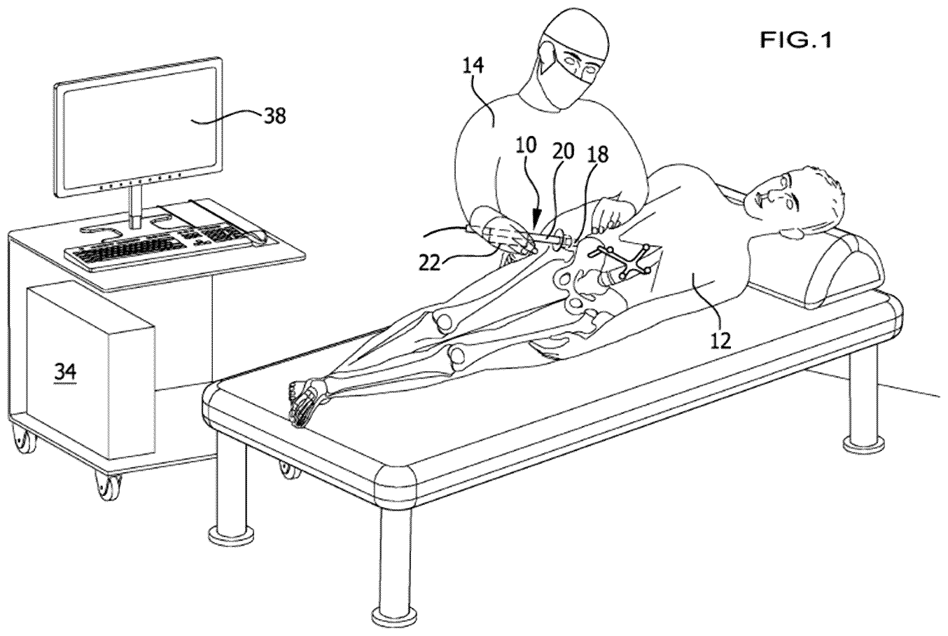

FIG. 1: a schematic perspective view of a device according to the invention, designed as an endoscopic device, as well as a user;

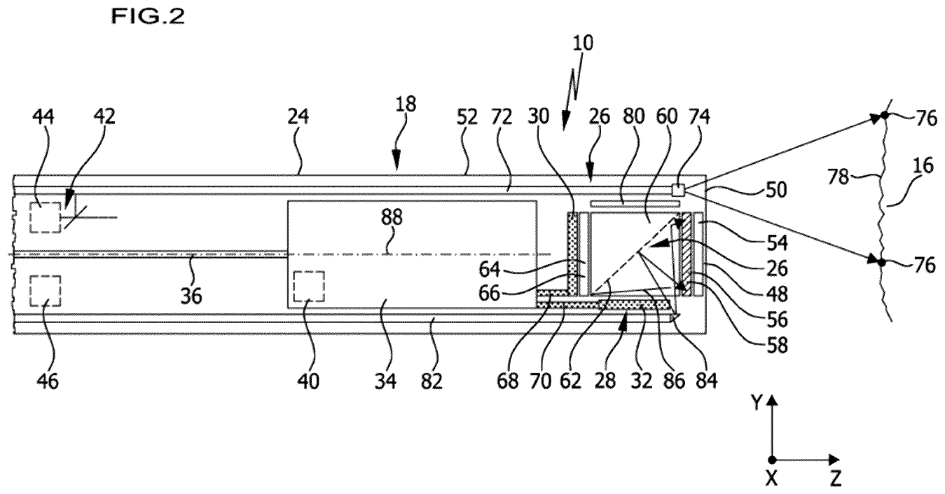

FIG. 2: a schematic representation of a distal end portion of the device of FIG. 1 as well as an examination object;

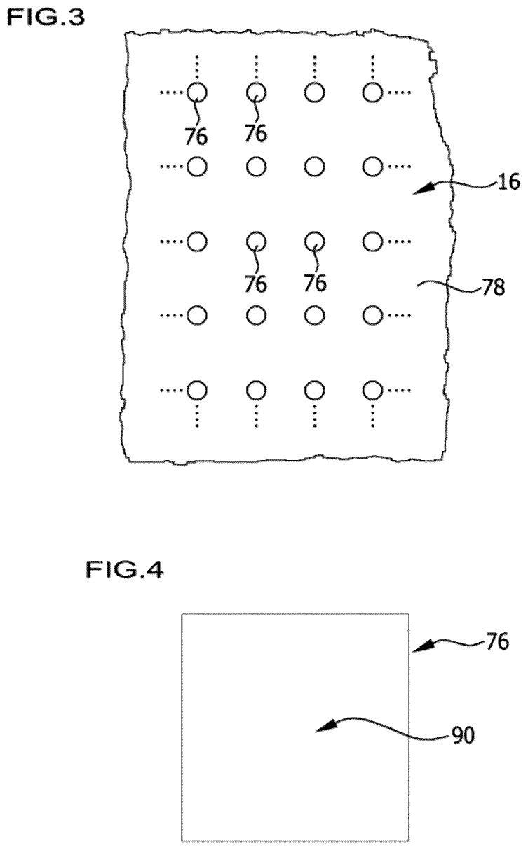

FIG. 3: schematically the object with a dot grid of raster light;

FIG. 4: a schematic representation of a luminous spot of the reference light on the object with speckle pattern;

FIG. 5: a luminous spot with speckle patterns at successive times, wherein an overlapping region of identical speckle patterns is schematically highlighted by a frame and arrow indicates a movement of the luminous spot;

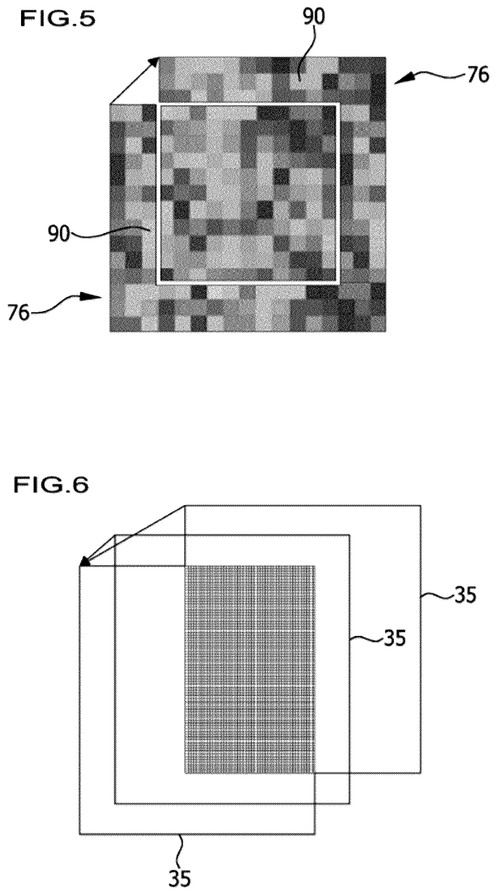

FIG. 6: three exemplary image data sets at successive time points and, hatched, an overlapping region contained therein, wherein arrows symbolically represent a shift of two image data sets depending on movement information back to the first image data set;

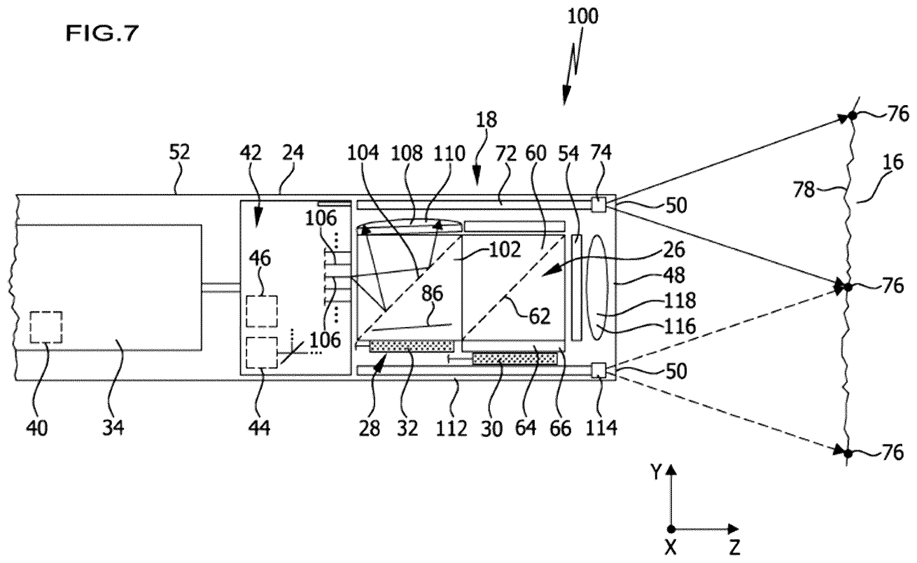

FIG. 7: a distal segment of a further preferred embodiment of the device according to the invention in schematic representation;

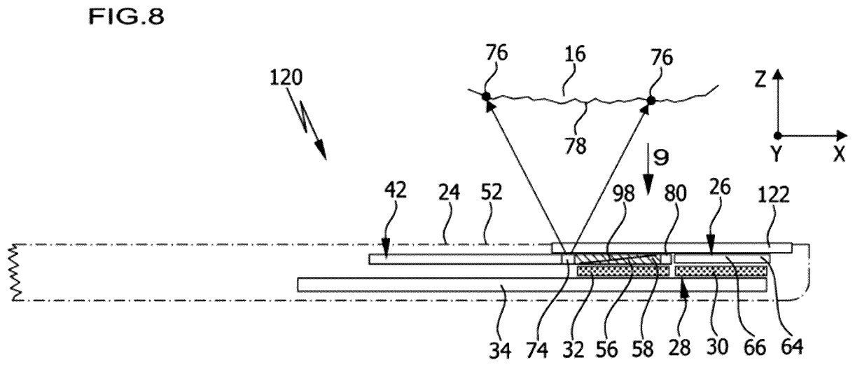

FIG. 8: a schematic representation of a further preferred embodiment of the device according to the invention;

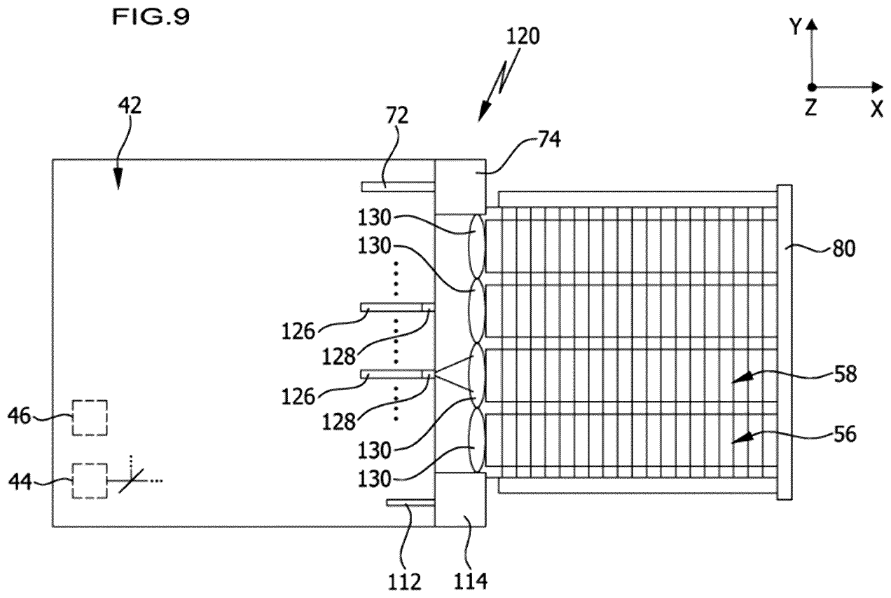

FIG. 9: a schematic view of the device of FIG. 8 in the direction of the arrow “9”;

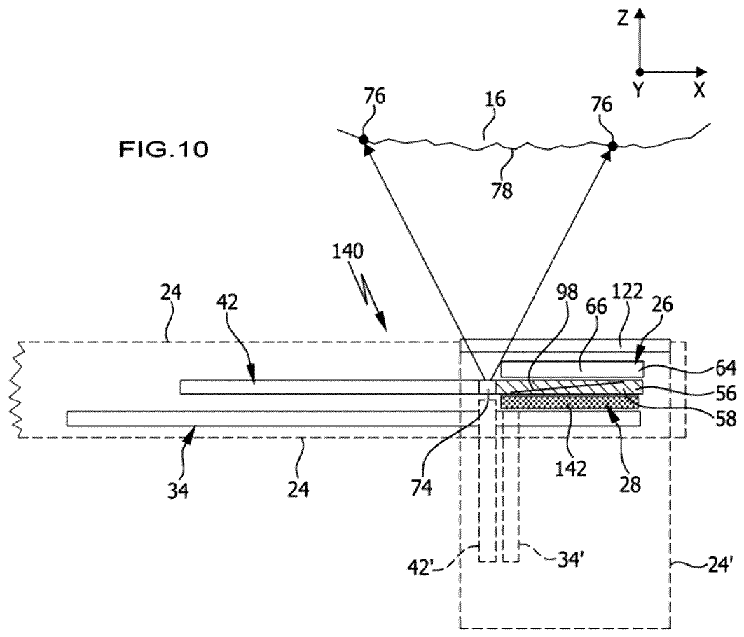

FIG. 10: a schematic partial representation of a further preferred embodiment of the device according to the invention;

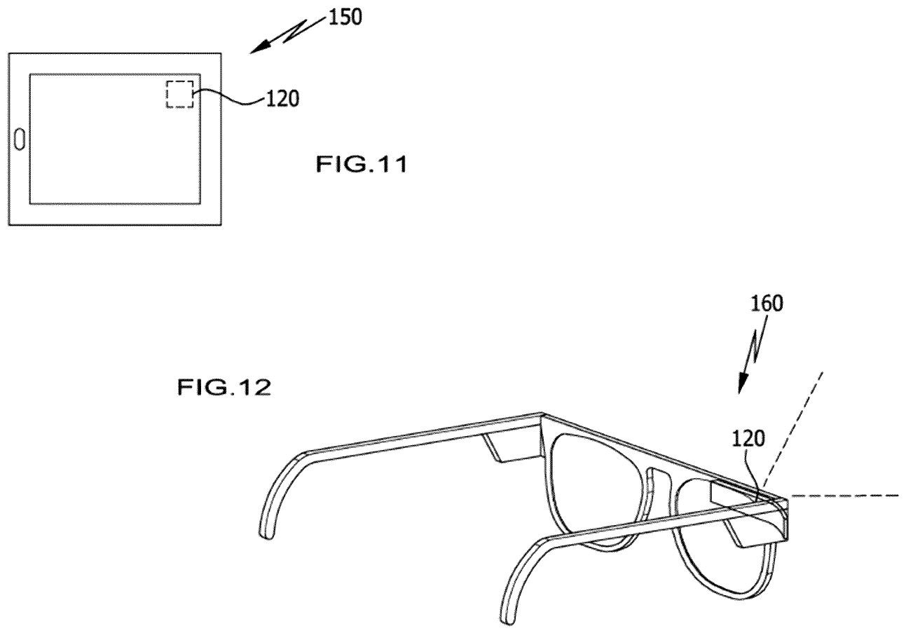

FIGS. 11 and 12: schematic representations of preferred embodiments of the invention.

With the advantageous embodiments of the device according to the invention explained below, a preferred embodiment of the method according to the invention can be practiced.

The invention is explained below, first of all, using the example of FIGS. 1 to 5 and the embodiment of the invention in FIGS. 1 and 2. The advantages mentioned in this context also apply to the preferred embodiments of the invention explained subsequently, so that reference can be made in this respect to the explanations given initially below. Only the essential differences between the various embodiments will be explained.

Identical reference signs are used for features and members that are the same or have the same effect.

FIG. 1 shows in schematic representation a preferred embodiment of the device according to the invention with the reference sign 10. The device 10 is in particular an endoscopic device which is used in the present example for examining a patient 12. The device 10 is hand-held and hand-guided and is operated by a user 14.

It is understood that the device 10 is shown only by way of example in an application in medical technology. The invention can also be used endoscopically to examine non-living objects. Furthermore, it should be emphasized that even when the device 10 is used in a medical environment, it can only be used for informational purposes instead of diagnostic purposes.

In the present case, the device 10 is guided through a body opening into the interior of the patient's body 12 in order to optically examine an object 16 (FIG. 2). In this case, a 3D surface data set of the object 16 is to be generated.

The device 10 comprises a distal segment 18 that is inserted into the interior of the body, and a proximal segment 20. For example, a handle element 22 for the user 14 is arranged at the proximal segment 20.

In the present embodiment, the distal segment 18 comprises a casing 24, in particular a shank-shaped casing, for accommodating components of the device 10. There is in particular the advantage of a high degree of structural integration for achieving a compact structure in which numerous components are arranged in the casing 24. In particular, the plurality of components may be arranged at the distal segment 18.

The device 10 comprises an optical unit 26 arranged in the casing 24, a sensor unit 28 arranged in the casing 24 and comprising two image sensors 30, 32, and further at least one data processing unit 34. In the present example, the data processing unit 34 is arranged and shown in the casing 24.

Image signals from the image sensors 30, 32 can be fed to the data processing unit 34 and processed by it. For this purpose, the data processing unit 34 is designed and programmed to perform corresponding calculations. For this purpose, an application program can be stored executably in the data processing unit 34 or in a memory unit connected thereto.

A data processing unit 34, which may or may not be part of the device 10, may alternatively be positioned outside the casing 24 (FIG. 1). It may be envisaged, for example, that information is only partially processed or preprocessed in the data processing unit 34 inside the casing and is transferred to the further data processing unit 34 outside the casing, this data processing unit 34 carrying out a further evaluation or detailed evaluation. A line 36 may be provided for data transmission and is preferably a high-speed data line.

For example, a display unit 38 may be controlled by the at least one data processing unit 34 to display the scene. The display unit 38 may be part of the device. Furthermore, a memory unit 40 for storing data sets of the device may be provided, which is formed separately from or integrated into the data processing unit 34.

or integrated therein.

Data transmissions between components of the device 10 and/or a data transmission with external components, for example the display unit 38 or an evaluation unit, may be wireless and/or cabled.

The device 10 further comprises an illumination unit 42 for providing light, as explained below in particular raster light and reference light. To this end, the illumination unit 42 comprises a light source 44, which may preferably be disposed in the casing 24 and more particularly in the distal segment 18.

Alternatively, the light source 44 may be arranged in the proximal segment or even separately, in which case a light guide may be provided for supplying the light.

The light source 44 is a laser light source. Via a beam splitting element, raster light and reference light can be separated and coupled into the respective optical path.

Furthermore, the illumination unit 42 may comprise a light source 46 for providing object light. The light source 46 is not implemented in the device according to FIG. 2 and is therefore only shown schematically. However, it could also be present as in the embodiments explained below.

An inlet opening 48 and an outlet opening are formed at a distal face of the casing 24, the inlet opening 48 preferably being arranged centrally and the outlet opening 50 in the present example being arranged on an outer wall 52.

The optical unit 26 comprises, in the direction of incidence of the light originating from outside, a filter element 54, in particular an IR filter, and adjoining it a volume phase hologram (VPH) 56. The VPH 56 is designed, for example, as a transmissive diffraction grating 58. The VPH 56 can reflect under the Bragg condition and act as a concave mirror for the reference light as explained below.

In the direction of incident light, the VPH 56 is followed by a beam splitter element configured as a beam splitter cube 60. The beam splitter cube 60 includes a reflective layer 62 for reflecting the raster light. In contrast, light of the visible spectrum is transmitted by the reflection layer 62.

Instead of the beam splitter cube 60, a different type of wavelength-sensitive beam splitter element can be used, for example a beam splitter plate.

Visible light passes through the beam splitter cube 60 and can reach the image sensor 30. Upstream of the image sensor 30 in the direction of the incident light, the optical unit 26 includes an optical element 64 that is phase and/or amplitude modulating. In the present example, the optical element 64 is a microlens array 66. Via the microlens array 66, the observed scene is imaged onto the image sensor 30.

The image sensor 30 is in operative connection with the data processing unit 34 via a signal line 68.

The NIR light reflected from the beam splitter cube 60 reaches the image sensor 32. The image sensor 32 is operatively connected to the data processing unit 34 via a signal line 70.

The illumination unit 42 comprises a light guide 72 for supplying raster light from the light source 44. The light guide 72 may comprise, for example, a rod and/or a fiber and may be rigid or flexible in configuration.

The raster light is guided to an optical uncoupling element 74 at the exit aperture 50. The uncoupling element 74 is, for example, a planar hologram for creating a pattern of luminous spots 76 (spots) on the surface of the object 16.

Instead of using a planar hologram as the uncoupling element 74, a spatial light modulator (SLM) may be provided, for example. The modulator may preferably be arranged in the distal segment 18 or also in the proximal segment 20. For example, the modulator is used when a depth range of the device 10 is delimited and an increased light intensity is required per luminous spot 76.

In the present case, the reference light comprises light of the NIR and/or visible wavelength range from about 700 nm or less to 1300 nm. Preferably, the spectrum comprises a plurality of discrete wavelengths in a wavelength window of about 1 nm to 10 nm in width. For example, the wavelength spacing is approximately 1 nm.

FIG. 3 schematically depicts the luminous spots 76 of the dot pattern on the surface 78 of the object 16. A respective luminous spot 76 comprises, for example, a size of approximately 100 μm. The grid size is, for example, approximately 1 mm and, as mentioned, can depend on the working distance.

The raster light reflected from the surface 78 passes through the filter element 54 and the VPH 56, and the raster light is reflected at the reflection layer 62 toward the image sensor 32. It may be provided that the reflection layer 104 is polarizing and/or that the optical unit 26 comprises a polarizing optical element.

The image sensor 32 is optimized with respect to its sensitivity to light in the spectral range of raster light. Any back reflections from the surface of the image sensor 32 that pass through the reflection layer 62 may be absorbed by an absorber element 80 on the opposite side from the image sensor 32.

In addition to the raster light, in-package reference light from the light source 44 is used. In the present example, the reference light downstream of the aforementioned beam splitter element (for example, a fiber coupler) is guided toward the distal end of the casing 24 via a light guide 82. The light guide 82 may be in the form of a rod or fiber, rigid or flexible. At the end, the light guide 82 comprises an optical uncoupling element 84, in this case designed as a reflector and in particular as a prism reflector.

Reference light is emitted from the uncoupling element 84, preferably with a spherical wavefront, in the direction of the reflection layer 62. Reference light is reflected from the latter into the VPH 56.

In the present case, the respective Bragg condition for reference light in the VPH 56 is satisfied. Reference light is diffracted back from the VPH 56, wherein an essentially planar wavefront 86 is formed. Here, for evaluation reasons, the system is advantageously designed such that the wavefront is inclined relative to a plane defined by the surface of the image sensor 32.

The wavefronts 86 (only one shown) of the different wavelengths in the reference light comprise a different slope from each other due to the respective Bragg condition.

In the image sensor 32, the raster light interferes with the reference light. The related image signals are transmitted to the data processing unit 34.

The data processing unit 34 can create a three-dimensional dot grid from the interfering signals of the raster light and reference light in the manner described preceding, using digital optical holography. A reference data set in this regard is indicative of lateral information and depth information and is provided by the data processing unit 34. “Lateral” is to be understood in the present context as referring in particular to transverse and preferably perpendicular to an axis 88 defined by the device 10.

The dot grid is used to provide support points for the reregistration of the image data set of the surface 78, as explained below. In a sense, the dot grid may scan the surface 78 at the points as an envelope surface of the object 16.

Provision may be made for the light guides 72, 82 to be arranged in the transverse direction of the device 10, presently perpendicular to the drawing plane, approximately centrally of the beam splitter cube 60 to facilitate propagation of the spherical wave in the direction of the VPH 56.

The reference data set comprises lateral information about the luminous spots 76 as well as absolute depth information. The depth information is obtained from the phase differences of the plurality of wavelengths used in the raster light and reference light.

The object light of the object 16 reaching the image sensor 30 is converted into image signals and supplied to the at least one data processing unit 34. The at least one data processing unit 34 generates an image data set 35 (FIG. 3). The image data set 35 can be spatially registered by the data processing unit 34 with respect to the reference data set, thereby providing a 3D surface data set of the surface 78.

Image data sets 35 obtained with the object light are subject to blurring due to noise. For this reason, in practice, for example, an otherwise possible maximum resolution of the image data set 35 cannot be achieved with the device 10. A typical, maximum resolution may be located in the region of approximately 25 μm, for example.

In order to improve the image data sets 35 and in particular to provide a high-resolution 3D surface data set, it is possible in the device 10 to relate the reference data set to the image data sets 35 as explained below in order to improve the image quality. For this purpose, a relative movement of the device 10 and the object 16 is used, wherein this relative movement can be determined via the device 10 itself. A separate device for determining the relative movement is not required.

It proves advantageous that the trembling of the hand-held and hand-guided device 10 itself, which inevitably occurs in practice, is used for determining the relative movement.

When the coherent raster light impinges on the surface 78, the luminous spot 76 is formed. As a result of the rough surface texture, the luminous spot 76 comprises a speckle pattern 90, which is shown schematically in sections in FIG. 4 as an intensity image. Here, for example, dark segments symbolize high intensity and light segments symbolize low intensity.

During relative movement of the device 10 and the object 16, the luminous spot 76 is moved over the surface 78. The speckle pattern 90 remains stationary due to the nature of the surface 78, but is displaced within the luminous spot 76. This is indicated in FIG. 5, wherein a white frame schematically encloses the overlap.

The speckle pattern 90 can be examined in a time-dependent manner. Through this speckle correlation of successive speckle patterns 90, the data processing unit 34 can produce movement information in the lateral direction with high accuracy that is indicative of relative movement.

Relative movement in the depth direction may be determined by the possibility of absolute depth information based on the use of the multiple wavelengths in the raster light and reference light.

As a result, the data processing unit 34 can generate movement information in the lateral direction and depth direction.

The image data sets 35, which are obtained by means of the object light, can be evaluated on the basis of the movement information in such a way that overlapping regions can be assigned to one another. This is shown schematically in FIG. 6, which shows three image data sets 35 and one overlapping region.

Since the relative movement of the device 10 and the object 16 also shifts the image information in the image data sets 35, the inverse movement can be applied to an image data set 35 to bring it spatially into matching with a previous image data set 35. In this way, the overlapping regions of the image data sets can be located within the scene.

By computationally integrating the two or more successive image data sets, the signal-to-noise ratio in the image data sets can be improved as the noise contained in the respective image data set 35 changes over time. Integration may also be considered to be averaging, as mentioned above.

It is understood that a high frame rate is advantageous. For example, frame rates of 250 Hz or more may be preferred, preferably more than 1 kHz. The frame rate should be selectable high enough to allow identification of overlapping regions within successive image data sets as large as possible.

In particular, it is advantageous if more than two image data sets can be smoothed together.

As a result, the data processing unit 34 can provide a 3D surface data set of the object 16 without loss of detail and while maintaining higher spatial frequencies. The 3D surface data set is high resolution and preferably resolved to the resolution limit of the system.

With noise-free or essentially noise-free computed image data set 35 or 3D surface data set, it is particularly possible to further increase the resolution computationally by applying deconvolution operations. Alternatively or complementarily, “super-resolution” data sets can be created, for example, by interpolation.

As mentioned earlier, depth information can be obtained in absolute terms. This provides, for example, the possibility of providing spot-dependent movement information for individual luminous spots 76. For example, different lateral trans-formation information may be present at the different luminous spots 76, which can be taken into account when computing the image data sets. For example, the surface 78 of a moving, “breathing” object 16 can be measured in this way. For this reason, the device 10 according to the invention is particularly suitable for application to the examination of moving objects 16, for example inside the body.

3D surface data sets can be combined to form an overall scene (stitching). Here, there is preferably no or only minimal mismatch between the individual data sets.

Assuming that the image sensors 30, 32 are each essentially square with an edge length a, a specifically compact design of the device 10 can be achieved at the distal segment 18. The optical unit 26 with the beam splitter cube 60 is approximately determined in the region of the image sensors 30, 32 by their dimensions. Thus, the entrance area of the aperture is approximately a2 and the volume is approximately a³ (lower limits). Overall, a dimension transverse to the axis 88 of preferably less than about 1.5 a² in cross-section and even more favorably less than about 1.25 a² can be obtained.

FIG. 7 shows a distal segment 18 of a device according to the invention assigned the reference character 100 in a preferred embodiment. Compared to the device 10, the device 100 comprises in particular the difference that no VPH 56 is used for the reference light. Instead, in addition to the beam splitter cube 60, another optical element in the form of a beam splitter cube 102 is used.

Differing from the device 10, the image sensors 30, 32 are positioned differently. In the device 10, the image sensors 30, 32 define planes aligned at an angle of essentially 90° and in a sense frame the beam splitter cube 60 and the microlens array 66.

In contrast, the planes of the image sensors 30, 32 in the device 100 are parallel or coincident. The beam splitter cubes 60, 102 are arranged laterally adjacent to the image sensors 30, 32 and associated with a respective image sensor 30, 32.

In the device 100, the beam path for the object light is reversed relative to the device 10. The object light is reflected at the reflection layer 62, whereas the raster light is transmitted. In the beam splitter cube 102 downstream of the beam splitter cube 60 in the direction of arrival, the raster light is reflected at a reflection layer 104 in the direction of the image sensor 32.

Reference light is uncoupled via an uncoupling element 106 having an essentially spherical wavefront. The uncoupling element 106 is directed toward the back side of the reflection layer 104. From the reflection layer 104, the reference light reaches an optical element 108, in this case in the form of a concave mirror 110. A substantially planar wavefront 86 is provided via the concave mirror 110, as in the case of the device 10 by means of the VPH 56.

The reference light may be coupled out via an optical waveguide forming or comprising the aforementioned beam splitter element. Raster light and reference light are separated. The reference light is coupled out depending on the wavelength at the plurality of side-by-side arranged uncoupling elements 106. An optical waveguide may be provided, for example the last-mentioned waveguide comprising the uncoupling elements 106 arranged in parallel side by side.

It may be provided that the reflection layer 104 is polarizing and/or that the optical unit 26 comprises a polarizing optical element. When the reflection layer 104 is polarizing, all of the raster light may be reflected onto the image sensor 32. Generally, the polarization of the emitted and/or received raster light can be adjusted to substantially suppress unwanted reflections from the optical unit 26.

Due to the use of multiple wavelengths in the reference light, as mentioned, multiple uncoupling elements 106 arranged side-by-side are provided (indicated in FIG. 7). This results in wavelength-dependent differently inclined wavefronts 86, as is the case with the device 10.

The reference light and the raster light are provided, for example, by an integrated illumination unit 42 in which the light source 44 is arranged.

FIG. 7 shows in the device 100 a further light guide 112 with an uncoupling element 114 arranged thereon in the region of the exit opening 50. In the present example, these components correspond in structure and arrangement to the light guide 72 and the uncoupling element 74 and are preferably arranged near a casing wall, wherein the light guides 72, 112 can receive the optical unit 26 and the image sensors 32 between them.

The uncoupling element 114 may also be, for example, a planar hologram. Via the two uncoupling elements 74, 114, the object 16 can be illuminated with two patterns of luminous spots 76. In order to be able to separate the contributions of the respective pattern, the illumination can be activated, for example, with a time delay and the recording can be synchronized via the image sensor 32, respectively.

Object light can optionally also be provided and coupled out via the device 100. For this purpose, the light guide 112 is applied, for example, without an uncoupling element 114.

The device 100 according to FIG. 7 further comprises an optical element 116 in the form of a converging lens 118 on the input side for pre-focusing and for increasing the wide angle of the device 100.

In the device 100, instead of the converging lens 118, a curved microlens array can be used to increase the wide angle, for example in the form of a spherical shell.

FIGS. 8 and 9 show a schematic representation of an advantageous embodiment of the device according to the invention, designated by the reference 120. The device 120 is characterized in particular by a flat structure in which the extension in one direction, in this case the height direction in the drawing, is substantially smaller than in the directions oriented transversely thereto.

For this reason, the device 120 is particularly suitable for installation in a portable electronic device, for example a smartphone or a tablet computer.

Assuming square image sensors 30, 32 with respective channel length a, the light entrance area is 2 a². The mounting height can be achieved to a maximum of a/4, for example, and thus the optical volume can be achieved to a maximum of approximately a³/2 (lower limits). This estimation is made assuming an edge length of about 8 mm for the image sensors 30, 32 and a build-up thickness of the components of about 1 mm to 2 mm.

In the device 120, the image sensors 30, 32 are arranged in a common plane. Upstream in the Direction of arrival, the microlens array 66 is positioned in front of the image sensor 30. Arranged in front of the image sensor 32 is the VPH 56. The VPH 56 and the microlens array 66 are preferably located in a common plane.

An entrance window 122 is provided on the entrance side, covering the VPH 56 and the microlens array 66.

The illumination unit 42 may preferably be arranged at least partially in the same plane laterally adjacent to the VPH 56 or the microlens array 66. The data processing unit 34 is arranged, for example, on the side of the image sensors 30, 32 facing away from the entrance side. The image sensors 30, 32 may be integrally designed on the data processing unit 34, for example.

The emission of raster light may take place via the uncoupling element 74, which may be directly or indirectly coupled to the illumination unit 42.

The device 120 may in particular omit the beam splitter cubes 60, 102.

For example, an optical waveguide is arranged in the illumination unit 42 for separating the reference light and the raster light from each other. Reference light is supplied for coupling, for example, via light guides 126. An uncoupling element 128 is arranged on the respective light guide 126, from which the reference light emits preferably with a spherical wavefront. The light guides 126 and uncoupling elements 128 are preferably arranged side by side in the plane of the VPH 56.

The reference light is incident on microlenses 130 arranged in the same plane and positioned side-by-side in series, respectively, providing a respective planar wavefront that propagates through the VPH 56. When the respective Bragg condition for the different wavelengths in the VPH 56 is satisfied, the reference light is diffracted toward the image sensor 32.

As in the previous cases, the wavefronts 86 of the individual wavelengths are inclined relative to each other and preferably inclined relative to the plane of the image sensor 32.

Light that does not meet the Bragg condition of VPH 56 due to diffraction or scattering may be absorbed by the absorber element 80.

An advantage in using reference waves via the array of microlenses 130 or a functionally equivalent multifocal element is that a planar wave of reference light of nearly constant intensity can be coupled out across the plane of the VPH 56. In this way, a sufficient signal yield can be obtained even at the edge of the image sensor 32.

Instead of the array of microlenses 130, a plurality of VPHs may be additionally employed, each performing the function of one of the microlenses 130. These VPHs could be integrated into an optical element that could, for example, be physically and optically coupled directly to the illumination unit 42 and the VPH 56. In this way, any gaps, such as in microlenses 130, could be avoided, thereby providing optimized wavefields.

Alternatively, for example, an elongated wave field can be generated that impinges laterally on the VPH 56. Arrays of GRIN lenses, for example, are used here. In this way, a phase front extending along the VPH 56 can be provided continuously so that no phase jumps occur in the transition to the VPH 56.

The side-by-side placement of the image sensors 30, 32 results in a depth-dependent parallax of the raster light and object light in the device 120. Due to the fact that the depth information can be determined absolutely, it is nevertheless possible to register the image data set 35 to the reference data set.

FIG. 10 shows with the reference sign 140 a preferred embodiment of the device according to the invention. The device 140 is highly integrated and designed in such a way that instead of two image sensors 30, 32 only one image sensor 142 is used. This image sensor 142 is suitable for recording both the NIR contributions of the reference light and raster light and the visible contributions of the object light.

The device 140 is also suitable for a flat configuration, for example, in a portable communication device or intracorporeal device, such as a stomach pill. Alternatively, a slim design is conceivable (reference signs with apostrophes).

On the entrance side, the entrance window 122 is provided, followed by the microlens array 66. Both the object light and the raster light pass through the VPH 56. The structure of the VPH 56 and the coupling of the reference light have already been described preceding in connection with the device 120 as well as the related modifications (FIGS. 8 and 9).

Light reaches the image sensor 142, the image signals of which are processed by the data processing unit 34.

In order to achieve the most compact structure possible in the height direction, the optical element 64 is necessary. However, this may prove troublesome to the propagation of the raster light.

To improve this, an amplitude mask with a structure that is wavelength-dependent can be used as optical element 64. For the object light, the structures can be reflective, whereas they are transparent for the raster light. In this case, the optical element 64 would be essentially transparent for the raster light.

Even in the case that amplitude or phase masks fully influence the raster light, this “disturbance” could be removed from the image signals for the raster light. The methods of deconvolution in this respect are known to the skilled person.

FIG. 11 shows a communication device according to the invention, designed as a smartphone, with the reference sign 150, with a device of the preceding type, for example the device 120.

FIG. 12 shows a head-mounted device according to the invention, designated by the reference sign 160, designed as data glasses, with a device of the preceding type, for example the device 120 or 140. Via the device 120 or 140, the immediate environment could be recorded with high precision in order to then either optimize this information or use it as a background for an “overlay”. Or as a background for an augmented reality (AR) overlay.

In the optimization approach, it would be conceivable to read a book with its 3D topography in such a way that the letters appear sharp and in exactly the same place. For this, one needs the topography of the page as object 16 and high-resolution letters. These can be superimposed on the field of view via the data glasses. The letters would be read sharply without any disturbing offset.

In the “overlay approach,” external information measured by the device 120 or 140 would be adjusted to actual reality. In the dental field, for example, a dental implant could be displayed at the exact correct position in the dentition, visualized to the dentist via 3D AR data glasses and, if necessary, shared via an external display unit 38.

### LIST OF REFERENCE SIGNS

10 Device
12 Patient
14 User
16 Object
18 Distal segment
20 Proximal segment
22 Handle element
24 Casing
26 Optical unit
28 Sensor unit
30, 32 Image sensor
34 Data processing unit
35 Image data set
36 Line
38 Display unit
40 Memory unit
42 Illumination unit
44 Light source
46 Light source
48 Inlet opening
50 Outlet opening
52 Outer wall
54 Filter element
56 Volume phase hologram (VPH)
58 Diffraction grating
60 Beam splitter cube
62 Reflection layer
64 Optical element
66 Microlens array
68, 70 Signal line
72 Light guide
74 Uncoupling element
76 Luminous spot
78 Surface
80 Absorber element
82 Light guide
84 Uncoupling element
86 Wavefront
88 Axis
90 Speckle pattern
100 Device
102 Beam splitter cube
104 Reflection layer
106 Uncoupling element
108 Optical element
110 Concave mirror
112 Light guide
114 Uncoupling element
116 Optical element
118 Converging lens
120 Device
122 Entrance window
126 Light guide
128 Uncoupling element
130 Microlens
140 Device
142 Image sensor
150 Communication device
160 Head-mounted device

1. An optical device for inspecting an object (16), comprising
a casing (24),
an optical unit (26) arranged in the casing (24) for incident light,
a sensor unit (28) with at least one image sensor (30, 32) arranged in the casing (24),
a data processing unit (34) which is coupled to the sensor unit (28) and evaluates image signals from the at least one image sensor (30, 32),
an illumination unit (42) arranged at least partially on or in the casing (24) for emitting raster light in the direction of the object (16),
wherein using raster light reflected from the object (16) and reference light internal to the casing, a three-dimensional dot raster and reference data sets indicative thereof for lateral information and depth information are provided by digital optical holography,
wherein a relative movement of the device (10; 100; 120; 140) and the object (16) is detected via the device (10; 100; 120; 140) and corresponding movement information in lateral and/or depth direction is generated,
wherein object light emanating from the object (16) is detected and a respective image data set (35) is generated successively in time, which is registered relative to the reference data set for generating a 3D surface data set,
wherein overlapping regions of two or more successive image data sets (35) are identified on the basis of the movement information and the overlapping regions are smoothed by integration of the respective image signals, in particular without loss of detail.

2. The device of claim 1, characterized in that overlapping regions of the two or more image data sets (35) are computationally superimposed by inverse movement.

3. The device according to claim 1, characterized in that a plurality of 3D surface data sets are assembled into an overall scene of the object (16), wherein boundaries between 3D surface data sets are determined based on the movement information.

4. The device according to claim 1, characterized in that, for determining the relative movement, at least one luminous spot (76) of the dot pattern with speckle pattern (90) generated by the raster light on the object (16) is examined as a function of time, wherein a displacement of the speckle pattern (90) within a luminous spot (76) is determined and movement information in the lateral direction is derived therefrom.

5. The device according to claim 1, characterized in that the raster light and the reference light comprise a spectrum with a plurality of discrete wavelengths, and in that movement information in the depth direction and/or absolute depth information can be determined from phase differences of the multiple wavelengths.

6. The device according to claim 1, characterized in that at least one of the following applies:
the raster light is or comprises infrared light and/or light of the visible spectrum;
the object light is or comprises light of the visible spectrum, in particular of a contiguous spectral range.

7. The device according to claim 1, characterized in that the illumination unit (42) comprises at least one light guide (72, 82, 112) for raster light in the casing (24) and an optical uncoupling element (74, 84, 114) arranged in particular distally on the casing (24), preferably that the uncoupling element (74, 84, 114) is or comprises a planar hologram, for fanning out the raster light into the dot pattern on the object (16).

8. The device according to claim 1, characterized in that at least one of the following applies:
the illumination unit (42) comprises a light source (44) for providing the raster light and the reference light, which is arranged in the casing (24);
the illumination unit (42) comprises a light source (46) for providing the object light, which is arranged in the casing (24).

9. The device according to claim 1, characterized in that the illumination unit (42) comprises at least one light guide (72, 112) for object light in the casing (24) and an optical uncoupling element (74, 114) arranged in particular distally at the casing (24).

10. The device according to claim 1, characterized in that a common image sensor (142) is provided which is sensitive to the spectrum of the object light and to the spectrum of the raster light, in particular that the sensor unit (28) comprises only one image sensor (142).

11. The device according to claim 1, characterized in that the sensor unit (28) comprises two image sensors (30, 32), wherein object light can be guided onto one of the image sensors (30, 32) and raster light can be guided onto the other image sensor (30, 32) via an optical element (60, 102, 116) of the optical unit (26), preferably that the image sensors (30, 32) are more sensitive to the respectively detected light than to the respectively other light.

12. The device of claim 11, characterized in that the optical element (60, 102, 116) is or comprises at least one wavelength sensitive beam splitter element (64, 102).

13. (canceled)

14. The device according to claim 1, characterized in that two image sensors (30, 32) are provided, which are positioned laterally adjacent to each other, particularly in a common plane, and are covered by at least one entrance window (122).

15. (canceled)

16. The device according to claim 1, characterized in that two image sensors (30, 32) are provided which are arranged in planes aligned at an angle to one another, and in that a wavelength-sensitive beam splitter element (60, 102) is arranged upstream of a respective image sensor (30, 32) in the direction of arrival of the object light or the raster light.

17. The device according to claim 1, characterized in that the optical unit (26) comprises an image sensor (30, 32) which is sensitive to object light and comprises a phase-modulating and/or amplitude-modulating optical element (64) upstream in the direction of arrival of the object light, in particular a microlens array (66), a phase mask or an amplitude mask.

18. (canceled)

19. The device according to claim 1, characterized in that the optical unit (26) comprises a VPH (volume phase hologram) (56) for diffraction of the reference light towards the at least one image sensor (30, 32, 142).

20. The device according to claim 1, characterized in that the illumination unit (42) for generating an essentially planar wavefront (86) of the reference light above a plane of the at least one image sensor (30, 32) comprises one of the following:
a plurality of microlenses (130) arranged side by side in a row;
a multi-volume hologram;
an array of GRIN lenses.

21. The device according to claim 1, characterized in that the optical unit (26) comprises an optical element (108) for expanding reference light having a planar or substantially planar wavefront (86) in the direction of the at least one image sensor (30, 32), in particular that the optical element (108) is a concave mirror (110).

22. (canceled)

23. The device according to claim 1, characterized in that the device (10; 100; 120; 140) is hand-held and/or hand-guided.

24. The device according to claim 1, characterized in that any one of the following applies:
the device (10; 100; 120; 140) is an endoscopic device which is at least partially insertable with the casing (24) into an examination object;
the device (10; 100; 120; 140) is or comprises a portable communication device (150), in particular a smartphone or a tablet computer;
the device (10; 100; 120; 140) is or comprises a head-mounted device (160).

25. (canceled)
